(19) United States
(12) Patent Application Publication
(10) Pub. No.: US 2003/0126770 A1
(43) Pub. Date: Jul. 10, 2003

(54) Integrated forming shoe having massage and shock-proof effects

(76) Inventor: Sung-Ho Chen, Chang-Hua Hsien (TW)

Correspondence Address: BACON & THOMAS, PLLC, 625 SLATERS LANE, FOURTH FLOOR, ALEXANDRIA, VA, 22314

(21) Appl. No.: 10/040,355

(22) Filed: Jan. 9, 2002

(51) Int. Cl. A61F 5/14

(52) U.S. Cl. 36/141; 36/25 R

(57) ABSTRACT

An integrated forming shoe having massage and shock-proof effects includes an arched and bulged section corresponding to the arch portion of human sole. The arched and bulged section has a profile and height matching the arch portion of the sole. A massage block is embedded in the interior of the insole corresponding to the arched and bulged section for stimulating the vital point reflection zone on the arch portion (i.e. Yung Chuan vital points) to attain massage and stimulation effect.

### FIELD OF THE INVENTION

The present invention relates to an improved shoe and particularly to an integrated forming shoe that has an arched and bulged section embedded a massage block corresponding to the Yung Chuan vital points (Burst Spring vital points) location of human sole to stimulate the reflection zone of vital points to achieve massage effect.

### BACKGROUND OF THE INVENTION

In the highly competitive working places and global slow economic conditions, most people work very hard and often work overtime to make a living. Exercises and rests have increasing been overlooked. People under such conditions for a prolonged period of time tend to feel fatigue easily, and are prone to suffer from various types of physical disorders. These disorders often are developed gradually and could be overlooked. If not treated or cured timely, people's health conditions could be deteriorated rapidly. Hence in recent years more and more people around the world are increasingly aware of better health care concept. Massage is one of simple and convenient health care exercises. As a result, shoe inserts that can stimulate vital points of sole have been developed and are widely accepted on the market. Referring to FIGS. 1 and 2 for shoes in the earlier time, the shoe insole 7 is flat and cannot touch the vital points on the arch of the sole. There is no massage effect.

Then there are massage insoles such as the one shown in FIG. 3. The insert 1 have a plurality of bulged massage granules 2 formed thereon and is adhered or stitched to the insole of the shoe. The massage granules 2 may contact the sole and generate massage effect. However, the massage effect is rather limited. As the arch of the foot (i.e. Yung Chuan vital points) cannot contact the massage granules 2, dead angles are formed. As the arch portion of the foot has a lot of vital point reflection zones, and is important to human body, not being able to contact these vital points seriously downgrades the massage effect. Moreover, to adhere or stitch the insert 1 is time-consuming and troublesome. After utilizing for a long period of time, the insert tends to breaking loose or hurting people's foot.

FIG. 4 illustrates a massage sandal, which has a plurality of toe bridges 4 formed on the toe section of the shoe insole 3. On the insole corresponding to the arch and heel of the foot, there are respectively a round-shaped bulged lump 5 and a fortress-shaped bulged lump 6. While the sandal thus formed can improve massage effect, the result is still not satisfactory. As the round-shaped bulged lump 5 is located on the arch position, the fortress-shaped bulged lump 6 is located on the heel and is not an ergonomic design. People's weight usually is support by the sole and heel, the round-shaped bulged lump 5 can contact the arch portion partially, but cannot reach the vast reflection zone on the arch area. Moreover, the fortress-shaped bulged lump 6 often has too high of elevation, thus restrict massage distance between the round-shaped bulged lump 5 and fortress-shaped bulged lump 6. As a result, massage effect cannot be fully developed.

Then there are sandals that have a length only about ⅔ of the foot length, and the arch of the foot is located on the end section of the sandals to get massage effect. When people wear these types of sandals, the heels do not have support from the insert. Wearing for a prolonged period of time, the foot could result in deformation and inflict not healthy condition to people. Moreover, people usually wear sandals for a limited of time in any day, and sandals are not suitable for wearing outdoors because of aesthetic concerns. All this indicates that there are still rooms for improvement.

### SUMMARY OF THE INVENTION

The primary object of the invention is to resolve aforesaid disadvantages. The invention provides an integrated forming shoe that has massage and shockproof effect. The shoe of the invention has an arched and bulged section on the insole surface corresponding to the arch portion (i.e. Yung Chuan vital points) of human sole and a massage block embedded in the arched and bulged section to stimulate the vital point reflection zone to achieve massage effect.

Another object of the invention is to make an integrated forming shoe that has massage and shockproof effects without adhering and stitching to save assembly time, and to achieve a greater durability for wearing.

The foregoing, as well as additional objects, features and advantages of the invention will be more readily apparent from the following detailed description, which proceeds with reference to the accompanying drawings.

### BRIEF DESCRIPTION OF THE DRAWINGS

FIG. 1 is a schematic view of a conventional shoe insole and human foot in use.

FIG. 2 is a schematic view of another conventional shoe insole and human foot in use.

FIG. 3 is a schematic view of a conventional massage sandal.

FIG. 4 is a schematic view of another conventional massage sandal.

FIG. 5 is sectional view of a shoe of the invention, partly cutaway.

FIG. 6 is a perspective view of a massage block of the invention.

FIG. 7 is a schematic view of the invention in use.

FIG. 8 is another schematic view of the invention in use.

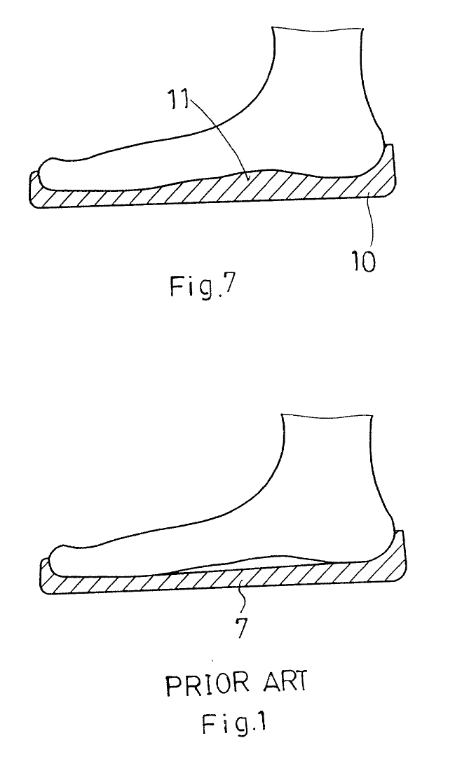

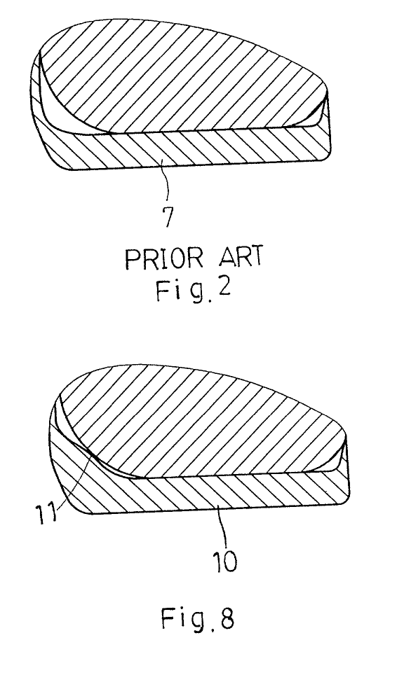

### DESCRIPTION OF THE PREFERRED EMBODIMENT

Referring to FIG. 5, the invention provides a shoe 10, which is designed to provide stimulation to the vital point reflection zone of the arch portion (i.e. Yung Chuan vital points) of human sole. The invention mainly utilizes an integrated forming process to make the shoe 10 which has an arched and bulged section 11 on the insole corresponding to the arch portion of human sole. The arched and bulged section 11 has a profile matching the arch portion of the sole, and has a height matching the arch portion to meet ergonomic requirements for giving people comfortable wearing without incurring ache. The arched and bulged section 11 is made from materials having a hard property. Inside the insole of the shoe 10 corresponding to the arched and bulged section 11, there is embedded a massage block 20 which is located under the arched and bulged section 11 to provide support to the sole and allow the arched and bulged section 11 to stimulate the arch portion (i.e. Yung Chuan vital points) of the sole to achieve massage effect.

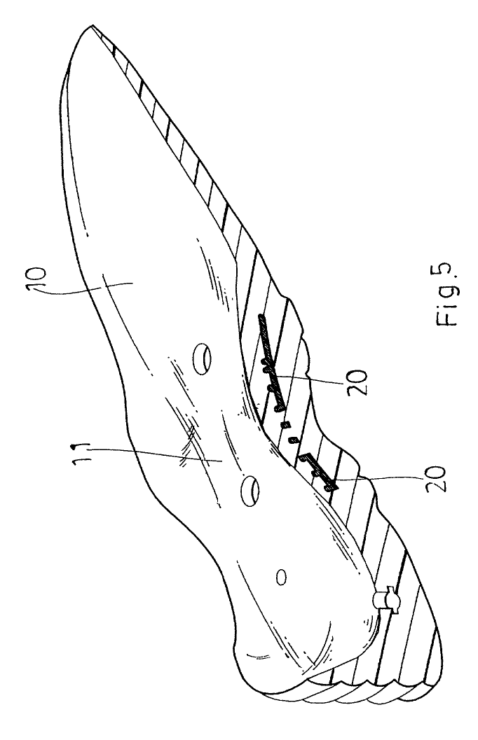

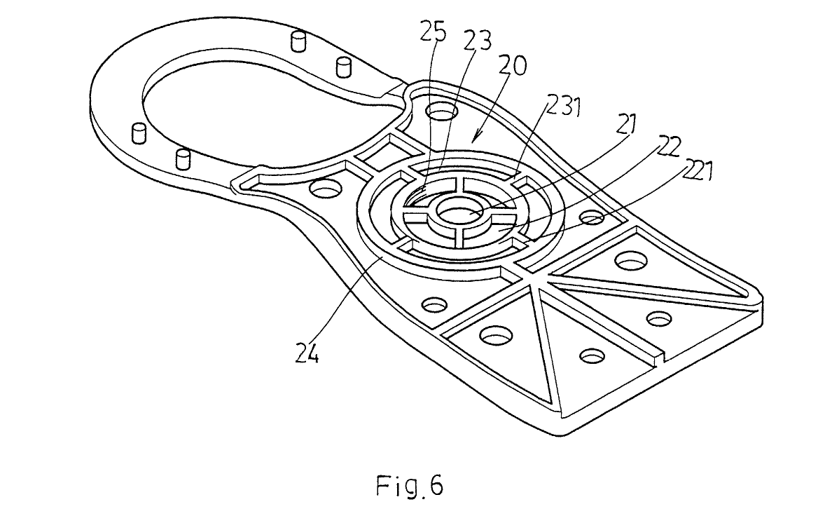

Referring to FIG. 6, the massage block 20 is integrally formed with the shoe 10 and is encased and sealed by the shoe 10. The massage block 20 has a center opening 21, an inner ring opening 22 and an outer ring opening 23. All the openings are filled with shoe materials. The massage block 20 has the property of SBS (styrene butadiene styrene). The massage block 20 further has an inner rib 221 and an outer rib 231 interconnecting with each other to give the massage block 20 desired elasticity. There is an annular ring 24 which has a perimeter greater than that of the massage block 20, and a step surface 25 with diameters changing gradually from large to small. When the top layer is under pressure and deformed, the lower annular ring 24 or lower layer step surface 25 absorbs the deformation of the massage block 20 and upper step surface 25, and provides massage effect.

Referring to FIGS. 7 and 8, when the shoe of the invention is worn on a human foot, the arch of the sole is located above the arched and bulged section 11, hence the tarsi of wear's foot may get desired support. The profile and height of the arched and bulged section 11 also match the vital point reflection zone of the sole. Thus the massage block 20 located under the arched and bulged section 11 can give the arched and bulged section 11 a thrust support to provide better stimulation effect to the sole. The shoe thus made also may last longer.

By means of aforesaid construction, the invention provides the following advantages:

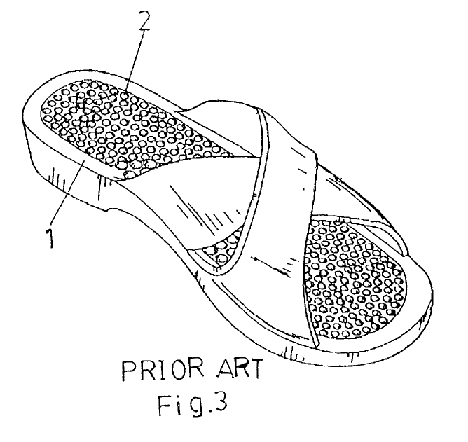

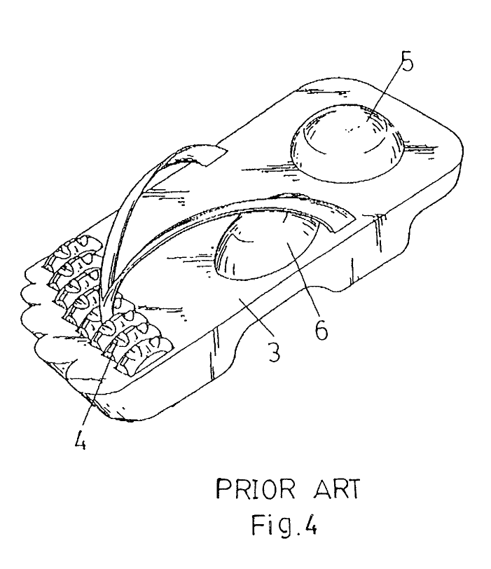

1. The shoe 10 and massage block 20 are integrally formed. Comparing with conventional insert 1 shown in FIG. 3, the invention can save adhering and stitching assembly time, and may be worn for a long period of time without breaking loose.

2. The arched and bulged section 10 can support the tarsi, and has a profile and height matching the arch of the sole, thus meets ergonomic requirements and may give wearers comfortable feeling.

3. The invention can provide stimulation to the vital reflection zones on the arch portion of the foot, therefore may give human vital functional organs desired protection to attain better health care.

4. The massage block resists deformation, and can achieve an improved massage effect, and is more reliable, and may be worn for a long period of time without damage.

What is claimed is:

1. An integrated forming shoe having massage and shockproof effects, comprising:
a massage block integrally formed with the shoe, the shoe having an arched and bulged section corresponding to the arch portion of a human sole.
